(19) United States
(12) Patent Application Publication
(10) Pub. No.: US 2013/0190788 A1
(43) Pub. Date: Jul. 25, 2013

(54) METHODS AND COMPOSITIONS FOR REPAIR OF VASCULAR TISSUE

(71) Applicant: Bruno Lowinger, Ann Arbor, MI (US)

(72) Inventor: Bruno Lowinger, Ann Arbor, MI (US)

(73) Assignee: COVALENT MEDICALS INC, SALINE, MI (US)

(21) Appl. No.: 13/643,472

(22) PCT Filed: Apr. 27, 2011
(86) PCT No.: PCT/US11/34157

§ 371 Date: Apr. 8, 2013

Related U.S. Application Data
(60) Provisional application No. 61/328,359, filed on Apr. 27, 2010.
(60) Provisional application No. 61/356,705, filed on Jun. 21, 2010.

(51) Int. Cl. A61B 17/12

(52) U.S. Cl. CPC A61B 17/12113
USPC 606/158; 606/200

(57) ABSTRACT

Processes for the correction or treatment of a vascular defect or vascularized tumor are provided including introducing an embolus formed of an adhesive to occlude an area of a defect or to prevent blood flow to a tumor. The inventive processes provide simple methods for long-term correction of vascular defects.

### CROSS-REFERENCE TO RELATED APPLICATIONS

This application depends from and claims priority to U.S. Provisional Patent Application Ser. No. 61/328,359 filed Apr. 27, 2010, and U.S. Provisional Patent Application Ser. No. 61/356,705 filed Jun. 21, 2010, the contents each of which are incorporated herein by reference.

### FIELD OF THE INVENTION

The present invention generally relates to closure of an embolization or tissue appendage using an adhesive sealant capable of bonding or sealing living tissues, and in particular, relates to use of a two-component composition that cross-links under surgical conditions with mechanical properties that are superior to those of undamaged tissue.

### BACKGROUND OF THE INVENTION

Correction of vascular defects is essential to maintaining proper blood flow to vital regions of the body. Vascular defects are compromised or abnormally utilized regions of vascular tissue resulting from a congenital abnormality, abnormally high vascular pressure, atherosclerotic disease, or from numerous other causes. In the case of atrial fibrillation, a normally essential portion of the vasculature such as the left ventricular appendage (LAA), a blind-ended, curved tubular LV (left ventricular) extension overlaying the LV anterior wall and adjacent left pulmonary veins, normally essential to decompress the LV when ventricular pressure is high, can suffer incomplete blood emptying leading to intraventricular clot formation. The defective emptying of the LAA results in severe complications for the subject.

Another example of a vascular defect is an aneurysm resulting from congenital defect or other disease, which results in a permanent, abnormal blood-filled dilatation or ballooning of a blood vessel. Aneurysms typically have thin walls that are vulnerable to rupture which could produce injurious pressure on surrounding tissue, impaired downstream blood flow, and death. Another example of a vascular defect is an arteriovenous malformation illustrated by a typically congenital shunt formed between an artery and a vein that often carries a substantial blood flow.

Embolization (the artificial blocking of blood flow) represents a minimally invasive procedure to treat vascular defects. The embolization of a vessel in an organ may be used to treat a variety of abnormalities. Illustratively, embolization may be used: 1) to control the bleeding caused by trauma; 2) to prevent profuse blood loss during an operation requiring dissection of blood vessels; 3) to obliterate a portion of or a whole organ having a tumor; or 4) to block the blood flow into abnormal blood vessel structures such as arterio-venous malformations and aneurysms.

Embolization is useful for treatment of cancerous and non-cancerous growths such as solid tissue tumors including uterine fibroids. Embolization may be performed by the delivery of embolic materials to the site of the vascular defect to occlude the defect. In the case of an aneurysm, a balloon is inflated over the neck of the aneurysm and a liquid embolic agent is introduced into the aneurysm. Embolic agents have also been used to occlude arteriovenous malformations. Accurate delivery of embolic agents has historically met with difficulty. In one of the more common procedures, a catheter is navigated to the site of the arteriovenous malformation and particles of polyvinyl alcohol with sizes selected for the particular application are introduced. This procedure requires guessing at the proper size of the particles and there is limited control over the placement of the particles, which upon release follow the path of greatest flow.

Other methods of treating vascular defects include introduction of an adhesive such as a n-butyle-2-cyanoacrylate that undergoes an exothermic polymerization reaction leading to vessel wall damage thereby forming a permanent plug. Similarly, methods that include introduction of ethanol are painful and also depend on formation of an occlusive thrombus to prevent blood flow through the area.

Treatment of cerebral aneurysms commonly involves an invasive clipping procedure that requires closing the base of the aneurysm using a specially designed clip with size and conformation characteristics that must be tailored for the site of defect, or use of endovascular coiling. The clip remains in the patient. Alternatively, endovascular coiling is used introduce a detachable coil (typically platinum) or latex balloons that are located to the site of the aneurysm and produce clot formation in an attempt to destroy the aneurysm. These methods both suffer from possible thrombotic complications as well as a possible need to repeat the procedure one or more additional times during a subject's lifetime. Difficulties with introduction of prior embolic agents include complications from the delivery method such as temporary blockade of flow through the vessel and the difficulty in controlling and containing the embolic agents, which allows some material to escape and block downstream vessels. In addition, prior art embolic agents commonly do not adequately adhere to the vessel walls often resulting in blood seepage. Biocompatible adhesives used in prior art procedures tended to adhere to the delivery equipment, resulting in a potentially fatal attachment of the delivery catheter to the embolic plug, or the formation of an extension of the embolic plug material as the delivery catheter is retracted.

Owing to the above-described limitations, there is a need for identifying more effective methods of closing vascular defects and materials or compositions for achieving successful correction.

### BRIEF DESCRIPTION OF THE DRAWINGS

FIG. 1 illustrates repair of a vascular defect such as a ventricular appendage according to one embodiment of the invention;

FIG. 2 illustrates repair of an arterial aneurism according by introducing biocompatible adhesive through a pericardial sheet that acts as a barrier (A), or by introducing biocompatible adhesive from the side of the barrier (B).

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

Processes and compositions are provided suitable for treatment of a vascular defect or a vascularized tumor. Processes for treating or correcting a vascular defect include introducing a biocompatible adhesive to a vascular defect such that the adhesive is blocked from accessing a bloodstream adjacent to the vascular tissue prior to cure of the adhesive, and inhibiting blood access to the defect by said introducing, thereby creating an embolus. In some embodiments, the adhesive is introduced to the luminal side of a defect. Optionally, the adhesive is introduced into the wall of the defect. The luminal side of the defect is optionally physically separated from the bloodstream by placing a barrier at the vascular defect proximal to the vascular wall wherein the barrier blocks the adhesive from assessing the bloodstream. Illustrative barriers include a clamp, staple, balloon, suture line, or structure. In some embodiments, flow of the bloodstream at the site of the defect or upstream therefrom is ceased, optionally prior to or simultaneous with introducing the adhesive.

A biocompatible adhesive optionally includes a cross-linkable protein in the form of a solution or suspension. A cross-linking agent solution optionally includes an aldehyde and an amino acid containing species reactive with the aldehyde. The aldehyde and the amino acid containing species are optionally present in a ratio between 20:1 and 1:1. The cross-linkable protein and the cross-linking agent active components are optionally present in a ratio of between 15:1 and 1:1. Upon combining the protein solution and cross-linking agent solution and allowing sufficient time for reaction to occur therebetween, a seal is formed capable of withstanding pressures of greater than physiological forces encountered. An amino acid containing species is optionally reacted with a multivalent aldehyde to form an oligomeric cross-linking agent. The amino acid containing species reactive with the multivalent aldehyde illustratively includes α-amino acids, β-amino acids, dipeptides, polypeptides, proteins, glycoproteins, and combinations thereof.

The cross-linkable protein is optionally in a solution. The cross-linkable protein is optionally recombinant. An adhesive as used in the processes described herein optionally includes a cross-linkable protein that is albumin, ovalbumin, casein, globulin, gelatin, or collagen.

An inventive process optionally includes contacting a structure with the tissue defect such that said structure is retained by said tissue adhesive sealant. A structure is optionally collagen. The tissue adhesive sealant is optionally simultaneously in contact with the tissue defect and a surgically implanted component.

Several defects are treatable by the processes illustratively including a defective or compromised ventricular appendage or atrial appendage, or a vascular aneurysm. Also provided are processes of treating vascularized tumor tissue in a subject's body including introducing a biocompatible adhesive to a site within an artery of a subject where the site is upstream of the tissue, and forming an emboli comprising the adhesive at the site. An occlusive structure is optionally deployed at the site, the structure contacting the adhesive to form at least a portion of the embolus. An occlusive structure is illustratively collagen, transplanted or autologous tissue, an aqueous suspension, platinum, a nickel titanium alloy, or combinations thereof.

Several tumors are treatable by the processes illustratively including a tumor located in a subject's uterus, liver, lung, spleen, breast, prostate, or brain. A tumor is illustratively a uterine fibroid.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The present invention provides processes for successful correction of vascular defects by introducing a biocompatible adhesive to the site of a vascular defect to successfully close the defect without the need for clot formation hence producing a far superior correction with reduced risk of complications. The invention has utility for the correction of vascular defects.

Inventive processes are provided that are operable to correct vascular defects or to treat a tumor. As used herein, the term “vascular defect” refers to compromised or abnormally utilized regions of vascular tissue resulting from a congenital abnormality, abnormally high vascular pressure, atherosclerotic disease, or from numerous other causes. Illustrative examples of a vascular defect include a vascularized tumor and an aneurysm. In some embodiments, a vascular defect arises due to disease or abnormality. Illustratively, a vascular defect is the result of atrial fibrillation where an atrial or ventricular appendage is no longer capable of properly regulating pressure within the associated heart chamber such that stagnant blood is preset within the appendage. Several embodiments of the present invention will be described with respect to treatment or correction of a heart condition by treating the atrial or ventricular appendage. It is appreciated that these processes are equally applicable to other vascular defects each of which are known to those of skill in the art and to the inventors.

Some embodiments of correcting a defect in vascular tissue include introducing a biocompatible adhesive into the defect such that the adhesive is blocked from accessing a bloodstream adjacent to said vascular tissue, and inhibiting blood access to said defect by said introducing. In some embodiments a vascular defect arises as the result of an atrial fibrillation such that the left ventricular appendage requires closure to prevent stagnant blood flow within the appendage. The left ventricular appendage (LAA) is a blind-ended, curved tubular left ventricular (LV) extension overlaying the LV anterior wall and adjacent left pulmonary veins. It functions to decompress the LV when pressure is high. Impaired LAA emptying may lead to blood stagnation inside the LAA and clot formation. Impaired LAA can be identified with pulse Doppler examination. LAA emptying velocities below 20 cm/s correlate with blood stagnation and thrombi formation.

Repair of a tissue defect such as an LAA or aneurysm includes introducing a biocompatible adhesive into the defect. A biocompatible tissue adhesive illustratively includes cyanoacrylates, and those detailed in U.S. Pat. Nos. 7,459,295; 7,351,426;7,141,428; 7,091,015; 7,083,634; 6,939,364; 6,875,427; 6,780,840; 6,773,699; 6,723,114; 6,596,318; 6,565,539; 6,500,427; 6,447,774; 6,310,036; 6,299,631; 6,251,370; 6,234,994; 6,136,341; 6,033,654; 5,980,866; 5,883,078; 5,817,303; 5,665,067; 5,464,471; 5,407,671; 4,909,251; 4,813,928; 4,735,616; 4,631,055; 4,600,574; 4,414,976; 4,377,572; 4,362,567; 4,359,049; and 4,298,598.

In some embodiments, the tissue adhesive is non-necrotic and is of high strength and variable cross-linking as detailed in U.S. Pat. No. 7,129,210. In some embodiments, a tissue adhesive includes a cross-linkable protein, and a cross-linking agent solution including an aldehyde and an amino acid containing species reactive with the aldehyde. In some embodiments, the aldehyde and amino acid containing species are present in a ratio between 20:1 and 1:1. In some embodiments, the protein and cross-linking agent are present in a ratio of between 15:1 and 1:1.

A biocompatible adhesive forms high strength seals and coatings with tissue masses or prosthetic materials through the cross-linking of an aqueous solution soluble protein with an oligomer formed by the reaction between an aldehyde and an amino acid containing species. The biocompatible adhesive has utility alone, or in combination with a structure material to form an embolus. The present invention further has utility to form a seal or a seal with a reinforcing structure thereover.

A cross-linkable protein according to the present invention is defined herein to include a protein capable of dissolving to form a solution or forming a suspension with a physiologically suitable aqueous solvent. Illustrative cross-linkable proteins include: ovalbumins; serum albumins; recombinantly expressed albumin illustratively expressed in rice such as Oryza sativa; albumin as described by Mawal et al. Biosci Rep., 1987; 7(1):1-9, optionally that of GenBank Accession No: BAF12524; and gelatins of human or animal origin from animals illustratively including horse, pig, cow, sheep, kangaroo, chicken, and fish. In some embodiments, a cross-linkable protein is human serum albumin isolated following expression in rice such as Oryza sativa which has the surprising property of differing cross linking relative to other human serum albumin. Suspension of collagen fibers is appreciated to be operative herein as a cross-linkable protein. In some embodiments a cross-linkable protein is albumin derived from or expressed in a plant such as rice. It is appreciated that recombinant whole or truncated proteins are operative herein so long as the recombinant proteins remain cross-linkable. Recombinant human serum albumin is operative as a cross linkable protein and the protein is described in U.S. Pat. Nos. 5,633,146; 5,986,062; 5,521,287; and 5,440,018. A recombinant protein is appreciated to lack viral, prion or bacterial contaminants associated with harvested proteins. An albumin operative herein may contain lesser amounts of other proteins or carbohydrates such as those found in blood plasma or elsewhere in a source organism. Human serum albumin is an illustrative cross-linkable protein operative in the present invention as utilized in the context of human tissue repair. It is further appreciated that ultrafiltration or other purification technique as applied to an albumin is successful in reducing the risk of immunological response or infectious agent introduction through the use of the present invention.

To form the first component of an inventive tissue adhesive sealant, a cross-linkable protein is dissolved in water or suspended in water to form a solution containing from 1 to 80 weight percent cross-linkable protein. In some embodiments, serum albumin is used as the cross-linkable protein from 1 to 55 percent by weight. In some embodiments, rice albumin is used as the cross-linkable protein, optionally from 20-65 percent by weight or any value or range therebetween, optionally 30-70 percent by weight, optionally 35-55 percent by weight. In some embodiments, aqueous solution proteins are present from 10 to 55 total weight percent. In some embodiments, aqueous suspension proteins are present from 0.3 to 9 total weight percent. Optionally, the cross-linkable protein is dissolved in an aqueous solution of physiologically acceptable buffer. Optionally, the protein is maintained in a dry or powder form until mixed with the cross-linking agent. Saline is an exemplary physiological buffer. Optionally, a cross-linkable protein solution includes an additive that illustratively includes an electrolyte, a thickener, an anti-microbial, a preservative, and a colorant. An electrolyte additive, if present, is optionally found in an amount that ranges from 0 to 5 total weight percent and illustratively includes sodium chloride, potassium chloride and sodium phosphate. A cross-linkable protein solution thickener according to the present invention is present from 0 to 50 total weight percent. Thickeners operative in the cross-linkable protein solution illustratively include sterilized collagen particulate, implantable grade fibrous materials such as polyamides, fluoropolymers and silk. A thickener in the present invention serves to modify the handling properties of the cross-linkable protein solution as well as to modify the mechanical properties of the resulting tissue adhesive seal. Other optional additives such as an anti-microbial, preservative and a colorant are those conventional to the art and are each present in an amount that typically ranges from 0 to 3 total weight percent. Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, New York and in The Merck Index: an encyclopedia of chemicals, drugs, and biologicals, 12th Edition, 1996, Merck & Co., Whitehouse Station, N.J. While it is appreciated that the viscosity of a cross-linkable protein solution according to the present invention is controlled through parameters that include cross-linkable protein concentration, the amount and identity of thickener, and the presence of various other additives. A cross-linkable protein solution viscosity is readily tailored to a specific task and has viscosity between that of water and 10,000 centipoise. In some embodiments, a cross-linkable protein solution has a viscosity sufficient to prevent runnage and therefore is generally in a range of between 10 and 1,000 centipoise.

A cross-linking agent solution component that upon combination with the cross-linkable protein solution forms a tissue adhesive includes a multivalent aldehyde and an amino acid containing species reactive therewith. The multivalent aldehyde is optionally a divalent aldehyde having a molecular weight of less than 1,000 Daltons. Optionally, the multivalent aldehyde has a C₀-C₁₆ alkyl or aryl backbone intermediate between two terminal aldehyde groups. Optionally, an aldehyde is a C₃-C₈ linear alkyl dialdehyde. Glutaraldehyde is an illustrative species of linear alkyl dialdehyde. It is appreciated that the introduction of a lesser quantity of a tri- or polyaldehyde with a majority of a dialdehyde creates cross-linkages within the cross-linking agent resulting in modified solution viscosity and final tissue adhesive mechanical properties. Optionally, a tri- or polyaldehyde is present at a stoichiometric molar ratio relative to a dialdehyde of 1:1000-1:30.

An amino acid containing species is reacted with a multivalent aldehyde to form an oligomeric cross-linking agent. The amino acid containing species reactive with the multivalent aldehyde includes α-amino acids, β-amino acids, dipeptides, polypeptides, proteins, glycoproteins, and combinations thereof. It is appreciated that both D- and L-conformers of a given amino acid are operative herein with the corresponding bioabsorbability associated with each conformer. It is appreciated that an amino acid containing species according to the present invention includes salts, esters and derivatized forms thereof. Additionally, where the amino acid is a β-amino acid, the resulting adhesive is comparatively resistive to bioabsorption. Derivatives to an amino acid containing species according to the present invention include solvation enhancing moieties such as hydroxyls, thiols, sulfonyls, halos; antibiotics; radioisotopes; magnetic markers; and antibodies. Optional amino acids include acidics such as glutamic and aspartic acid; aliphatics such as alanine, valine, leucine and isoleucine; and amides such as glutamine and asparagine. A optional amino acid containing species is shown in Formula I:

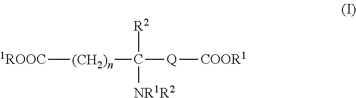

where Q is CH₂ or a nullity, R¹ is independently in each occurrence H, Na, K, C₂-C₆ alkyl; R² is independently H, C₁-C₂₀ alkyl group, a C₀-C₄ alkyl group having a substituent selected sulfonate, carboxylate, hydroxyl, quaternary amines, a radio isotopic ion, a magnetically detectable ion, an antibiotic moiety and an antibody; and n is an integer between 1 and 6 inclusive; hydrohalide salts thereof; and combinations thereof.

Some embodiments of the amino acid containing species of Formula I are L-glutamic acid, L-glutamic acid hydrochloride, sodium L-glutamate, potassium L-glutamate, monosodium L-glutamate, monopotassium L-glutamate, L-aspartic acid, L-aspartic acid hydrochloride, sodium L-aspartate, potassium L-aspartate, monosodium L-aspartate, and monopotassium L-aspartate, and combinations thereof. L-glutamic acid and L-aspartic acid are may be used owing to the resulting cross-linking efficacy. It is appreciated that monosodium L-glutamate, L-glutamic acid hydrochloride, monopotassium L-glutamate, monosodium L-aspartate, L-aspartic acid hydrochloride, and monopotassium L-aspartate into a cross-linking solution for a longer period of time yield similarly effective cross-linking solutions relative to L-glutamic acid.

According to the present invention, the amino acid containing species is present in the cross-linking agent solution in an amount such that the molar ratio of aldehyde moieties to amino acid or peptide subunits is between 20:1 and 1:1. It is noted that within this ratio range, an increase in amino acid containing species generally tends to increase the ultimate adhesive and cohesive strengths of the cured tissue adhesive sealant. Optionally, the aldehyde moieties to amino acid or peptide subunits molar ratio is between 10:1 and 4:1. Optionally, the ratio is between 8:1 and 6:1. In the instance where the aldehyde is glutaraldehyde and the amino acid containing species is L-glutamic acid, glutaraldehyde is optionally present from 2 to 40 weight percent of the solution with the amino acid containing species being introduced in an amount to satisfy the recited ratio. As with cross-linkable protein solution, the cross-linking agent solution optionally includes pH modifiers, surfactants, antioxidants, osmotic agents and preservatives. Examples of pH modifiers include acetic acid, boric acid, hydrochloric acid, sodium acetate, sodium bisulfate, sodium borate, sodium bicarbonate, sodium citrate, sodium hydroxide, sodium nitrate, sodium phosphate, sodium sulfite, and sulfuric acid. Surfactants operative herein illustratively include benzalkonium chloride. Antioxidants operative herein illustratively include bisulfates. Electrolytes operative herein illustratively include sodium chloride. Preservatives operative herein illustratively include chlorobutanol, sorbate, benzalkonium chloride, parabens, and chlorhexadines.

A tissue adhesive optionally includes a radio-opaque material so that an inventive process optionally includes subjecting the area of a malignancy to X-ray, nuclear magnetic resonance, or positron emission topography to determine the localization of the adhesive relative to a tumor or to normal tissue. Illustrative examples of radio-opaque materials illustrative include: barium salts such as barium sulfate, optionally halogenated barium salts, other heavy metal salts illustratively salts of bismuth, silver or lead; heavy metals embedded in silica filler added to the adhesive composition; organic iodine compounds illustratively methacylates; or halogenated polymers. In some embodiments, a radio-opaque material is iopromide, metrizamide, or mixtures and solutions thereof. Illustrative examples of radio-opaque materials are illustratively found in U.S. Pat. No. 4,866,132 and reference cited therein.

Methods of producing a tissue adhesive are illustrated in U.S. Application Publication No. 2009/0287313. In some embodiments, the preparation of a cross-linking agent solution typically begins with the mixing of the aldehyde into water at room temperature. The pH of the resulting solution is then assured to be between 2 and 11 and optionally raised to basic with an aqueous base such as sodium hydroxide. Optionally, the pH is increased to between 8 and 11. Optionally, pH is raised to between 8.2 and 8.8. Thereafter, sufficient solid L-glutamic acid is added to correspond to a final concentration of 0.2 molar upon full dissolution through mechanical agitation, sonication or passive dissolution. It is appreciated that variables such as the time allowed for dissolution, whether mixing occurs through agitation or sonication, the temperature of dissolution and subsequent filtering are all variables that are readily modified in the formation of a cross-linking agent solution. Proper control of these variables leads to a broad peak and high pressure liquid chromatography traces corresponding to a collection of large oligomeric species that are generally characterized in the case of glutaraldehyde-glutamic acid cross-linking agents as being hydrophilic and therefore having longer retention time on a C-18 column. This group of larger oligomeric species correlates with superior bonding properties in the cured inventive tissue adhesive sealant. Optionally, the final pH of the cross-linking solution is modified to be pH 1.5 to 9.0 prior to mixing with a cross-linkable protein solution. Optionally, the cross-linking agent solution is in a pH range of 1.5-4.5. It is appreciated that the gel time of the combined cross-linking agent solution and cross-linkable protein solution is varied as a function of cross-linking agent solution acidity. Generally, a more acidic cross-linking agent solution according to the present invention has a longer gel time than an otherwise identical cross-linking agent solution having a higher pH.

The biocompatible tissue adhesive is applied to the region of a vascular defect in a number of ways. By way of illustration, the two components that make up the tissue adhesive sealant may be quickly mixed together and then injected into a space formed by the tissue defect. An adhesive is optionally injected through the vascular wall into an interior space formed by the defect. A hole is optionally created through the wall of the vascular tissue. A hole is optionally 1, 2, 3, 4, 5, 6, 7, or more, in diameter or any value or range therebetween. A hole is optionally 5 millimeters in diameter. A hole is optionally created by use of an aortic hole punch such as that provided by Medtronic, Inc., Minneapolis, Minn. Optionally, a hole is created by a needle inserted into the defect to pass through the wall. The device used to create the hole is optionally the same device used to deliver a biocompatible adhesive. Illustrative adhesive delivery devices include catheters, syringes (with or without needles), hole punches, or other device known in the art.

In some embodiments, an adhesive is injected into a space formed by the defect from the intravascular region such as delivery via a catheter threaded into the interior of the associated vessel or chamber. This embodiment prevents the need for traversing the vascular tissue to deliver adhesive to the interior of a vascular defect.

An exemplary adhesive delivery device includes a proportional sized double-barreled syringe equipped with a mixing tip that delivers cross-linkable protein in a molar ratio relative to the cross-linking agent of between 15:1 and 1:1. Optionally, the cross-linkable protein is delivered at a molar ratio relative to the cross-linking agent of 8:1 and 1:1. Optionally, the cross-linkable protein is delivered at a ratio relative to the cross-linking agent of 5:1 and 3:1. Illustratively, the user attaches a mixing tip to a loaded syringe and by depressing the syringe plunger a mixed pre-gelled adhesive composition is urged from the mixing tip. Alternatively, a mixing tip is replaced by a spray nozzle tip, such as that sold under the trade name TISSEEL (Immuno AG, Vienna, Austria). With a spray nozzle fitted to the double-barreled syringe, an atomized spray of ungelled adhesive composition is released upon syringe plunger depression.

An inventive tissue adhesive composition is optionally delivered to a site of application as a three-component system including cross-linking agent, cross-linkable protein, and a structure material. Collagen is exemplary of structure materials used herein. Alternatively, transplanted or autologous tissue such as pericardial tissue may also be used. The structure material is optionally formed as an aqueous suspension that is delivered prior to, or in concert with, a cross-linking agent component and a cross-linkable protein component. Simultaneous delivery of a structure material is optionally facilitated by the use of a three-barreled syringe where the first and second barrels deliver cross-linkable protein and cross-linking agent as detailed above and the third barrel is loaded with structure material. Optionally, a mixing tip is provided with a triple-barreled syringe. Alternatively, a structure material suspension is intermixed with the cross-linkable protein component according to the present invention and delivered as a two-component system by way of a mixing or spray nozzle tip as detailed hereinabove. Optionally, a foaming agent is introduced into an adhesive component to facilitate the formation of a foamed tissue adhesive. A foaming agent operative herein includes tissue compatible surfactants. Illustrative of these foaming agents are non-toxic surfactants including, but are not limited to, fats or proteins in edible foams. However, the surfactant may be an ionic or non-ionic surfactant depending on the intended application. The ionic surfactants including, for example, anionic surfactants such as sodium stearate, sodium dodecyl sulfate, α-olefinsulfonate and sulfoalkylamides and cationic surfactants such as alkyldimethylbenzylammonium salts, alkyltrimethylammonium salts and alkylpyridinium salts; and amphoteric surfactants such as imidazoline surfactants. The non-ionic surfactants including, for example, polyethylene oxide alkyl ethers, polyethylene oxide alkylphenyl ethers, glycerol fatty acid esters, sorbitan fatty acid esters, sucrose fatty acid esters, and the like.

In situations where the inventive tissue adhesive composition is delivered in conjunction with a foaming agent, a propellant is optionally provided in fluid communication with a spray nozzle tip. Propellants illustratively include aerosol propellants such as carbon dioxide, nitrogen, propane, fluorocarbons, dimethyl ether, hydrochlorofluorocarbon-22, 1-chloro-1,1-difluoroethane, 1,1-difluoroethane, and 1,1,1-trifluoro-2-fluoroethane, alone or in combination.

A “structure” or “patch” is defined herein to include any shaped substrate compatible with surgical implantation and capable of being coated or impregnated by or cross-linked with an inventive sealant, shapes of which illustratively include a aqueous suspension, a solution, a powder, a paste, a sheet, a ring, a stent, a cone, a plug, a pin, a screw and complex three-dimensional shapes contoured to be complementary to specific anatomical features. Inventive structure materials illustratively include collagen; polylactic acid; hyaluronic acid; fluoropolymers; silicones; knitted or woven meshes of, for example, cellulosic fibers, polyamides, rayon acetates and titanium; skin; bone; titanium; stainless steel; these or other memory metals; or alloys such as nickel titanium alloys. Collagen is an illustrative structure material. Alternatively, pericardial or other body tissue may be used instead of a collagen structure. Optionally, the collagen is a flexible, fibrous sheet readily formed into a variety of shapes that is bioabsorbable and has a thickness of 2-5 millimeters. Such fibrous sheet collagen is commercially available from a number of suppliers. A collagen structure serves to enhance sealant strength while allowing some penetration of the inventive tissue sealant thereto. Optionally, in a surgical setting, a dry or a wetted absorbent gauze is placed proximal to the wound site in order to wick away any excess ungelled inventive tissue sealant prior to cure.

The adhesive may also be applied as a spray using, for example, the means described above or, alternatively a duel spray apparatus similar to the type disclosed by U.S. Pat. No. 4,792,062 or 6,722,532. In such an application, the cross-linkable protein in a solution and a cross-linking agent solution (as discussed above) are simultaneously delivered by a spray apparatus proximate to the intended target area resulting in the mixing of the components as an adhesive.

The adhesive may be bondable to metals optionally following the pretreatment of the metal with H₂O₂.

In the context of minimally invasive surgical procedures, the adhesive of the present invention is optionally delivered to a target bonding site using either a tip that mixes the adhesive components prior the reaching the ends of the catheter or that delivers the glue through two separate channels and mixes it at the end. Appropriate mixing tips are described above and are known in the art.

A biocompatible adhesive is delivered in sufficient quantity to at minimum coat the walls of a vascular defect such that forced contact of the walls will result in a closed defect. Optionally, adhesive is introduced into a defect or space formed by a defect in sufficient quantity to fill the space formed by the defect. The exact volume of adhesive alone, or combined with one or more structures is readily determined by one of ordinary skill in the art introducing the adhesive and relates to the size and shape of the defect. Optionally, an adhesive is introduced in sufficient volume that at risk of exiting the defect such as through the entry hole.

The introduction of the biocompatible adhesive is performed by a manner that prevents or blocks the adhesive from contacting the blood stream. Many methods are optionally used to prevent contact with the bloodstream such as with the use of a barrier. A barrier is optionally placed over the internal surface of the vascular tissue in the region of the defect, serves to clamp the base of the defect proximal to the vascular wall, or act as a suture, staple, or otherwise closure at the base of the defect proximal to the vascular wall, or functions by other method known or envisioned by one of skill in the art. In some embodiments, a barrier functions as a sheet providing physical or chemical separation between an biocompatible adhesive and the bloodstream. Introducing a biocompatible adhesive into a defect and allowing sufficient time for the adhesive to cure or otherwise adhere to the defect is sufficient to inhibit or otherwise prevent blood from accessing the defect from the intravascular side of the vascular tissue.

In some embodiments a barrier is placed over the defect in the interior of the vascular tissue that forms a space defined by the defect thereby separating this space from the nearby blood flow. A barrier is optionally in the form of a closed stent surface, a sheet such as a pericardial sheet, an occlusive structure, or other surface or material.

In some embodiments, a barrier is created by a clamp, staple or suture line that closes the base or other region of the defect proximal to the vascular tissue wall. Optionally, a clamp is applied to the outer surface of a defect proximal to the vascular wall to clamp or otherwise close the region of the defect proximal to the vascular wall. This clamping effectively separates the interior of a defect or region including a defect from blood access. An adhesive is then introduced into the interior of the defect filling the space formed by the clamping. Upon cure or adhesion of the adhesive, the space is sufficiently filled and adhered such that removal of the clamp prevents blood from accessing the interior of the defect or a sufficient portion thereof to produce a therapeutic effect.

An inventive process optionally includes ceasing flow of a bloodstream at the site of a defect prior to introducing an adhesive. Blood flow in a vessel or other vascular tissue is achieved by any method known in the art. Illustratively, a balloon is delivered to a region upstream or at the site of a defect. A balloon is optionally delivered by a catheter by minimally invasive techniques. The balloon is then inflated blocking flow of the bloodstream in the vicinity of the defect. The lack of blood flow optionally serves to improve the ability of the adhesive to cure or otherwise adhere to the defect. A ceased blood flow also prevents inadvertent access of the adhesive to the bloodstream reducing the chances of adhesive associated complications. A balloon or other device used to cease blood flow optionally serves as a barrier to prevent leakage of adhesive outside or beyond the defect. Other methods of ceasing blood flow at or near the defect are similarly operable.

In some embodiments, a balloon is inflated at the site of the vascular defect to act as a barrier to adhesive access to the bloodstream during introduction of an uncured adhesive. A balloon optionally acts as a barrier and simultaneously is inflated to a sufficient size to prevent blood flow at the site of the vascular defect during the process of correcting the defect. A balloon is optionally made from or is coated with a material that will not bind to a biocompatible adhesive so that the balloon may be deflated and more easily removed from the site of the vascular defect at the termination of a correcting procedure. Materials used to form a balloon optionally are polyvinyl chloride, or cross-linked polyethylene such as polyethylene terephthalate (PET) or nylon. Other suitable materials are also operable.

It is appreciated that when a barrier or structure is impregnated or coated with uncured adhesive that the coating or impregnating of the adhesive in the barrier or structure is sufficient to block free access of the uncured adhesive to the bloodstream during introduction.

In some embodiments a tissue adhesive is simultaneously in contact with the tissue defect and a surgically implanted component. A surgically implanted component optionally includes a stent, a barrier, a structure, a prosthetic, an exogenous portion of vascular tissue, a microbead, a coil, polyvinyl alcohol sponges (Ivalone), or other surgically implantable device. Optionally, a coil is introduced into the interior of the defect by endovascular therapy.

Illustratively, a platinum coil is delivered into the interior of the defect to partially or substantially fill the defect space. The coil is optionally used as a structure or barrier with remaining spaces filled by biocompatible adhesive. The adhesive both fills the remaining space, maintains the surgically implanted component within the defect, and provides sufficient strength and elasticity to resist complications and provide a therapeutic effect.

In some embodiments a component of the adhesive, barrier, or structure or combinations thereof may further be infused with a pharmaceutical agent such that the adhesive, barrier or structure functions as a drug delivery agent. The pharmaceutical agents that can be delivered by the present invention include organic, inorganic and organometallic compounds without limitation. The compounds may be water soluble or water insoluble. Further, pharmaceutical agents include beneficial agents that affect a cell, tissue, organ or body system, the body system illustratively including the nervous system, cardiovascular system, immune system, reproductive system, musculoskeletal system, lymphatic system, alimentary system, excretory system, endocrine system, hormone system and blood circulatory system.

Further, pharmaceutical agents which can be included illustratively include: an analgesic, an anesthetic, an anthelminthic, an anti-allergic, an anti-arrhythmic, an anti-asthmatic, an antibiotic, an anticonvulsant, an antidepressant, an anti-diabetic, an antifungal, an antihypertensive, an anti-inflammatory agent, anti-migraine, an anti-neoplastic, an anti-parasitic, an anti-tumor agent, an anti-ulcer agent, an antiviral, an anxiolytic, a bronchodilator, a cough or cold agent, a cytostatic, a hypnotic, a hypoglycemic, a metastasis inhibitor, a muscle relaxant, a neoplastic, a sedative and a tranquilizer compound. Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, New York and in The Merck Index: an encyclopedia of chemicals, drugs, and biologicals, 12th Edition, 1996, Merck & Co., Whitehouse Station, N.J.

Pharmaceutical agents deliverable with an adhesive, a barrier, a structure or combinations thereof are those with a molecular weight in the range from about 50 Daltons to about 10,000,000 Daltons.

Prodrugs are included as pharmaceutical agents. The term “prodrug” refers to compounds that are rapidly transformed in vivo to yield the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

In addition, it is intended that the present invention include compounds made either using standard organic synthetic techniques, including combinatorial chemistry or by biological methods, such as through metabolism.

The compositions optionally include an effective amount of the selected pharmaceutical agent in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to an individual along with the selected substrate without causing significant undesirable biological effects or interacting in a deleterious manner with any of the other components of the pharmaceutical composition in which it is contained.

A single pharmaceutical agent is delivered by the drug delivery device of the present invention. Optionally, two or more pharmaceutical agents may be delivered simultaneously by the drug delivery device of the present invention.

Also provided is a process of treating vascularized tumor tissue in a subject's body including introducing a biocompatible adhesive to a site within an artery of the subject where the site is upstream of the vascularized tumor tissue, and forming an emboli including the adhesive at the site.

As used herein the term “upstream” is meant as within the bloodstream of a blood vessel located at a region contacted by blood prior to the blood reaching the vascularized tumor tissue. These embodiments provide reduced or eliminated blood access to vascularized tumor tissue thereby starving the tumor of necessary nutrients and promoting death or shrinkage of the tumor tissue.

A biocompatible adhesive is introduced to a site by any method desired by one of skill in the art. In some embodiments, a biocompatible adhesive is introduced by way of an endovascular route such as by a catheter. The catheter is run through the circulatory system to the site of adhesive delivery. The adhesive is then introduced to the site to form an embolism that reduces or eliminates blood flow past the embolism.

In some embodiments an occlusive structure is introduced at the site. An “occlusive structure” as defined herein is a sheet, ring, plug or dam having sufficient thickness to serve as a barrier against blood transference therethrough. Inventive occlusive structure materials optionally include collagen; polylactic acid; hyaluronic acid; fluoropolymers; thermoplastics; silicones; knitted or woven meshes of, for example, cellulosic fibers, polyamides, rayon acetates and titanium; polypropylene; polyester; skin; bone; polyvinyl alcohol sponges (Ivalone); and metals or alloys such as platinum, titanium, or stainless steel. Collagen is an illustrative structure material. Alternatively, pericardial or other body tissue may be used instead of collagen. Optionally, the collagen is a flexible, fibrous sheet readily formed into a variety of shapes that is bioabsorbable and has a thickness of 0.5 to 10 millimeters, optionally 1 to 8 millimeters, optionally 2 to 5 millimeters. Such fibrous sheet collagen is commercially available from a number of suppliers. A collagen based occlusive structure serves to enhance adhesive strength while allowing some penetration of the tissue sealant into or onto the occlusive structure.

A tumor is optionally a cancerous or non-cancerous tumor. Exemplary tumors include solid tissue tumors. More specific illustrative tumor types include tumors of the uterus, liver such as hepatocellular carcinoma, lung, spleen, breast, prostate, or brain. In some embodiments, a tumor is a uterine fibroid. In some embodiments a tumor is a liver tumor. A tumor is optionally a breast tumor. A tumor is optionally the result of metastasis from a primary location, or is a primary tumor. Any degree of vascularization is operable to be treated by the inventive method. Treatment of a tumor optionally includes introducing 1, 2, 3, 4, 5, 6, 7, 8, 9, or more emboli to reduce or eliminate blood flow through the vasculature to a desired degree.

As used herein the term “subject” is defined as a human, non-human primate, equine, bovine, murine, or other animal with a vascular structure.

A generalized procedure for treating vascularized tumor tissue includes introducing a biocompatible adhesive into the body of a subject. A catheter is introduced via usual procedures to a chosen site in a mammalian body. Illustrative examples of a site include a Fallopian tube, a urethral or bile duct, a vascular site such as the hepatic artery or portal vein, or other site appreciated to benefit from formation of one or more emboli. It is generally desirable to utilize the largest inner diameter catheter practical in approaching the chosen site. The bolus of premixed or unmixed biocompatible adhesive is then introduced into the catheter and injected at the chosen site. Because the biocompatible adhesive becomes nonsoluble upon polymerization to forms the occluding mass, the precursor is optionally introduced slowly so to form an aggregate near the catheter distal tip. More than one injection of precursor is possible using this technique. Once the mass is formed, the catheter is removed.

In some embodiments an occlusive structure is coated with, impregnated with, or otherwise contacted with biocompatible adhesive. The occlusive structure is optionally delivered to a site at the end or through a catheter. The biocompatible adhesive polymerizes at the site forming a reinforced embolus preventing blood flow through or around. In some embodiments, additional biocompatible adhesive is introduced at the site prior to, simultaneous with, subsequent to, or combinations thereof, with the introduction of an occlusive structure. An embolus created by this method may provide additional securement or increased size of the embolus.

The present invention is further illustrated by the following examples that are intended to be illustrative of particular embodiments of the present invention. These examples are not intended to limit the scope of the present invention as defined by the appended claims.

### Example 1

### Preparation of Cross-linking Agent Solution for Use in a Biocompatible Adhesive

Fourteen grams of glutaraldehyde is added to 86 grams of deionized, distilled water with mechanical stirring. The resulting solution is titrated with aqueous sodium hydroxide to a pH of 8.5. Three grams of L-glutamic acid is added to the solution and allowed to mix for 72 hours until all of the added glutamic acid has dissolved.

### Example 2

### Closing a Left Ventricular Appendage

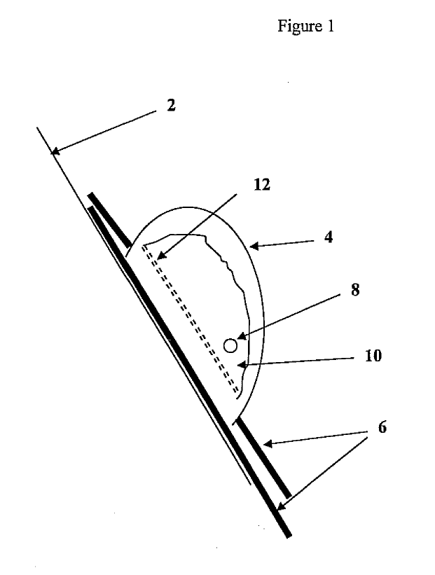

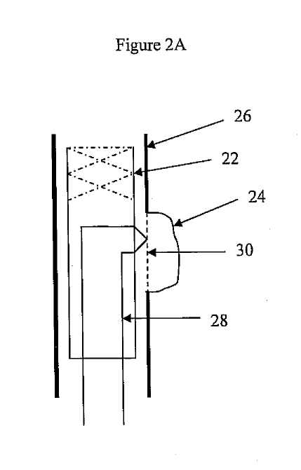

The heart tissue in the area of the left ventricular appendage is accessed by open surgical technique. As illustrated in FIG. 1, a surgical clamp 6 acting as a barrier is placed on the ventricular appendage 4 as close to the ventricular wall 2 as possible thereby assuring the desired area of the appendage 4 is segregated from the blood flow within the ventricle. A sharp pointed scalpel is used to make a pointed incision large enough to insert a hole punch size 3-5 mm. A hole 8 is then punched in the appendage. Several biocompatible adhesives 10 are tested for adequacy: 1) CovaMed™ Surgical Adhesive CovaBOND™ produced as detailed in U.S. Pat. No. 7,129,210; 2) cyanoacrylates such as INDERMIL® which is based on n-Butyl cyanoacrylate, or adhesives based on fibrin. As an optional step, a pericardium sheet or any other form of scaffold 12 is placed up against the clamp, staple or suture line prior to inserting the adhesive that acts as an additional barrier for the adhesive and as a scaffold for the ventricle tissue on the opposite side of the adhesive to promote healing of the ventricle wall. The adhesive is introduced using a short mixing tip for an open heart procedure or a long flexible or rigid mixing tip for a MIS (Minimally Invasive Surgery) by placing the tip in the hole and inserting a sufficient amount of adhesive in to the appendage to completely block any blood from entering the appendage once the adhesive is set and the clamp is removed.

A pressure-monitored water infusion system is constructed using I.V. tubing segments, an aneroid manometer, three-way stopcocks, and a balloon angioplasty pressure generator (Scimed Pressure Generator: Minneapolis, Minn.). The water infusion system is connected to the appendage by a needle inserted near the clamp opposite the hole into which the adhesive is introduced.

By stopcock manipulation, repeated trials of pressure-monitored, left ventricular appendage distensions are performed while checking the hole or region into which the needle is inserted for any fluid leakage. The pressure of the adhesive of U.S. Pat. No. 7,129,210 registers 0.9 bar (675 mmHg, 13.1 psi) without visible fluid leakage from the hole or from the opposite side of the clamp. Thus, the adhesive successfully bonds the inside of the appendage with sufficient strength and flexibility to resist pressure created by ventricular contraction.

The process is repeated using minimally invasive technique to access. Similar results are obtained.

### Example 3

### Effect of Cross-linkable Protein on Adhesive Strength

The process of forming the cross-linkable protein solution of above is repeated three different times. In each instance, bovine serum albumin is replaced by one of: human serum albumin, ovalbumin, and gamma globulin in like quantities. Thereafter, the process of Example 2 is repeated using each of these cross-linkable protein solutions separately as a component of the sealant according to the procedure in Example 2. Each of the sealants based on human serum albumin, ovalbumin and gamma globulin allowed for the repeated application of left ventricular distension pressures exceeding 2 atm before and after overnight storage in 4° C. water.

### Example 4

### Sealant Efficacy in Porcine Liver Model

A fresh porcine liver is excised and coupled by way of the hepatic artery to a pressurized plasma solution reservoir. Other vessels were sutured and the liver pressurized to 200 torr. A 10-12 mm core is excised from the liver to simulate a gunshot. A drop in pressure and hemorrhage of plasma is noted. A collagen occlusive structure having an outer diameter of 10 mm is coated with the tissue adhesive of U.S. Pat. No. 7,129,210 and the plug inserted into the liver bore. Within 3 minutes the liver supports a coupled reservoir pressure of 150 torr. Accordingly, it will be appreciated that the adhesive is operable to glue the collagen plug in place.

### Example 5

### Sealant Efficacy in Aneurism Model

A carotid artery having an internal diameter of 4 mm is stripped from a freshly slaughtered pig. The artery is coupled at one end to a plasma reservoir and a septum added to seal the other end. The artery is pressurized to 200 torr. A 1 mm transmural circular defect is simulated by a sharp excision of a tissue cylinder. A drop in pressure and hemorrhage of plasma is noted. A 3 mm diameter collagen sheet is pushed through the artery with a catheter and lodged in the excision. The tissue adhesive of U.S. Pat. No. 7,129,210 is delivered through the catheter tip. After the sheet has been held in place for 5 minutes, the catheter is removed and the artery is again able to withstand pressurization to pre-excision values.

### Example 6

### Aneurism Repair by Administration of Biocompatible Adhesive

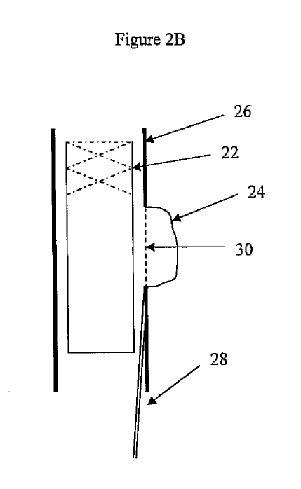

An aneurism present in an arterial wall created as in Example 5 is repaired by delivery of a biocompatible adhesive to the aneurism from the direction of the vessel lumen. As illustrated in FIG. 2A, a stent 22 is delivered by minimally invasive technique to the site of an arterial aneurism 24 in an arterial wall 26. A catheter 28 is manipulated to the site of the aneurism 24. A pericardial sheet 30 is positioned at the site of the aneurism 24 to act as a barrier preventing access of the adhesive to the bloodstream as well as promoting healing of the aneurism following delivery of the adhesive embolus. The 3 mm diameter pericardial sheet 30 used as a barrier is pushed through the artery with a catheter 28 and lodged at the base of the aneurism 24. The tissue adhesive of U.S. Pat. No. 7,129,210 is delivered through the catheter and deposited through the catheter tip to the space of the aneurism 24 to form an embolus. After the sheet 30 has been held in place for 2 to 5 minutes, the catheter is removed and the artery is again able to withstand pressurization to pre-aneurism values.

### Example 7

### Aneurism Repair by Administration of Biocompatible Adhesive

The procedure of Example 6 is repeated in a different animal by introducing the biocompatible adhesive under the pericardial sheet via access to the defect from the side of the sheet. As illustrated in FIG. 2B, a stent 22 is delivered by minimally invasive technique to the site of an arterial aneurism 24 in an arterial wall 26. A catheter 28 is manipulated to the site of the aneurism 24. The catheter 28 is either positioned at the edge of the aneurism 24 or extends to the area within the aneurism 24. A 3 mm diameter pericardial sheet 30 is positioned at the site of the aneurism 24 and either overlays the catheter 28 at its tip, or extends to the location of the catheter tip, to act as a barrier preventing access of the adhesive to the bloodstream prior to cure as well as promoting healing of the aneurism following delivery of the adhesive embolus. The procedure is optionally performed by traversing the sheet 30 by the catheter through a flap or self closing access point. The pericardial sheet 30 used as a barrier is pushed through the artery with a catheter 28 and lodged at the base of the aneurism 24. The tissue adhesive of U.S. Pat. No. 7,129,210 is delivered through the catheter 28 and deposited through the catheter tip to the space of the aneurism 24 to form an embolus. After the sheet 30 has been held in place for 2-5 minutes, the catheter is removed, and the artery is again able to withstand pressurization to pre-aneurism values.

### Example 8

### Arterial Aneurism Repair by Administration of Biocompatible Adhesive

The process of Example 2 is also used to correct a brain aneurism in a pig arterial aneurism model. An aneurism is created in the common carotid artery of a pig by methods similar to the pancreatic elastase-digested arterial sac (EDASA) models of Abruzzo, et al., AJNR Am J Neuroradiol, 1998; 19:1309-1314. The aneurism is corrected by the same procedure to produce an adhesive embolus capable of withstanding physiological forces successfully strengthening the area of the artery wall and preventing blood access into the aneurism.

Any patents or publications referenced herein are hereby incorporated by reference to the same extent as if each individual reference was explicitly and individually incorporated herein by reference. These patents and publications are indicative of the level of skill in the art to which the invention pertains.

It is appreciated that one skilled in the art will note modifications and variations in the invention as described herein. These modifications and variations that are equivalent to, and within the spirit of the present invention, are intended to be encompassed within the appended claims.

1. A process of correcting a defect in vascular tissue comprising:
introducing a biocompatible adhesive to said defect such that said adhesive is blocked from accessing a bloodstream adjacent to said vascular tissue prior to cure; and
inhibiting blood access to said defect by said introducing.

2. The process of claim 1 further comprising placing a barrier at said vascular defect proximal to the vascular wall wherein said barrier blocks said adhesive from assessing said bloodstream.

3. The process of claim 2 wherein said barrier includes a clamp, staple, or suture line.

4. The process of claim 1 further comprising ceasing flow of said bloodstream prior to said introducing.

5. The process of claim 1 wherein
said biocompatible adhesive comprises:
a cross-linkable protein; and
a cross-linking agent solution comprising an aldehyde and an amino acid containing species reactive with said aldehyde, said aldehyde and said amino acid containing species being present in a ratio between 20:1 and 1:1 and said protein and said cross-linking agent are present in a ratio of between 15:1 and 1:1.

6. The process of claim 5 wherein said cross-linkable protein is in a solution.

7. The process of claim 5 wherein said cross-linkable protein is a recombinant protein.

8. The process of claim 5 further comprising the step of contacting a structure with the tissue defect such that said structure is retained by said tissue adhesive sealant.

9. The process of claim 8 wherein said structure comprises collagen.

10. The process of claim 8 wherein said tissue adhesive sealant is simultaneously in contact with the tissue defect and a surgically implanted component.

11. The process of claim 1 wherein said defect is a ventricular appendage, an atrial appendage, or a vascular aneurysm.

12. A reinforced tissue capable of withstanding vascular pressures greater than 240 torr obtainable by the process of claim 1.

13. A process of treating vascularized tumor tissue in a subject's body comprising:
introducing a biocompatible adhesive to a site within an artery of said subject where said site is upstream of said tissue; and
forming an emboli comprising said adhesive at said site.

14. The process of claim 13 further comprising introducing an occlusive structure at said site, said structure contacting said adhesive to form said embolus.

15. The process of claim 14 wherein said occlusive structure comprises collagen, transplanted or autologous tissue, an aqueous suspension, platinum, or a nickel titanium alloy.

16. The process of claim 13 wherein said adhesive comprises:
a cross-linkable protein; and
a cross-linking agent solution comprising an aldehyde and an amino acid containing species reactive with said aldehyde, said aldehyde and said amino acid containing species being present in a ratio between 20:1 and 1:1 and said protein and said cross-linking agent are present in a ratio of between 15:1 and 1:1.

17. The process of claim 16 wherein said cross-linkable protein is in a solution.

18. The process of claim 16 wherein said cross-linkable protein is a recombinant protein.

19. The process of claim 16 wherein said cross-linkable protein is albumin, ovalbumin, casein, globulin, gelatin, or collagen.

20. The process of claim 13 wherein said tumor is located in a subject's uterus, liver, lung, spleen, breast, prostate, or brain.

21. (canceled)
